(19) United States
(12) Patent Application Publication
(10) Pub. No.: US 2024/0282406 A1
(43) Pub. Date: Aug. 22, 2024

(54) ARRAY-BASED TARGETED COPY NUMBER DETECTION

(71) Applicant: Illumina, Inc., San Diego, CA (US)

(72) Inventors: Yong LI, San Diego, CA (US); Youting SUN, San Diego, CA (US); Sidney KUO, San Diego, CA (US)

(73) Assignee: Illumina, Inc., San Diego, CA (US)

(21) Appl. No.: 18/438,619

(22) Filed: Feb. 12, 2024

Related U.S. Application Data
(60) Provisional application No. 63/486,038, filed on Feb. 20, 2023.

(51) Int. Cl. G16B 20/10

(52) U.S. Cl. CPC G16B 20/10

(57) ABSTRACT

Array-based targeted copy number detection, for instance detection on contaminated and/or variable concentration samples, includes obtaining a collection of intensity signals from assays of a set of input samples, performing a cross-sample calibration on the intensity signals based on reference sample(s), which calibration includes constructing a reference signal distribution based on intensity signals of the reference sample(s) and for one or more input samples calibrating a set of intensity signals corresponding to the input sample based on the reference signal distribution, determining, for the one or more input samples, and from a respective one or more calibrated sets of intensity signals corresponding to the one or more input samples, a respective at least one aggregated calibrated signal from targeted genomic region(s) to produce a collection of aggregated calibrated signals, and detecting variant(s) in the targeted genomic region(s) based on the collection of aggregated calibrated signals.

### BACKGROUND

Copy number detection can be performed with multiple types of arrays for genotyping, cytogenetics, or methylation. The Infinium BeadArray technology, as one example, offered by Illumina, Inc., San Diego, California, supports copy number detection in two modes: a discovery mode and a targeted mode. For the discovery mode, copy number variation/variant (CNV) events can be detected in an unbiased way in unknown regions of the genome, while in the targeted mode copy number change (i.e., CNV) detection is focused on specific genomic regions of interest.

CNVs are involved in many types of human diseases, such as neuropsychiatric disorders, developmental disorders, cardiovascular diseases, autoimmune diseases, and cancer, as examples. As a result, copy number detection assays have been useful in clinical applications, such as cytogenetics, carrier screening, pharmacogenomics, and precision medicine. Copy number detection also proves useful in veterinary genetics and other non-human genetics applications.

### SUMMARY

Shortcomings of the prior art are overcome and additional advantages are provided through the provision of a computer-implemented method. The method includes obtaining a collection of intensity signals from assays of a set of input samples including genetic material, and performing a cross-sample calibration on the intensity signals of the collection of intensity signals based on one or more reference samples. The performing the cross-sample calibration includes: constructing a reference signal distribution based on intensity signals of the one or more reference samples; and for one or more input samples of the set of input samples: obtaining a respective set of intensity signals, of the collection of intensity signals, corresponding to that input sample, the set of intensity signals corresponding to the input sample including (i) a first subset, C, of intensity signals from one or more targeted genomic regions of interest and (ii) a second subset, B, of intensity signals from at least one genomic regions outside the one or more targeted genomic regions of interest, and calibrating the intensity signals in C based on the reference signal distribution, to produce a respective calibrated set of intensity signals corresponding to the input sample. The method additionally includes determining, for the one or more input samples, and from a respective one or more calibrated sets of intensity signals corresponding to the one or more input samples, a respective at least one aggregated calibrated signal from the one or more targeted genomic regions of interest, wherein the determining produces a collection of aggregated calibrated signals, and detecting one or more variants in the one or more targeted genomic regions of interest based on the collection of aggregated calibrated signals.

Further, a computer system is provided that includes a memory and a processor in communication with the memory, wherein the computer system is configured to perform a method for improved calling of copy number variants in a genomic sequence. The method includes obtaining a collection of intensity signals from assays of a set of input samples including genetic material, and performing a cross-sample calibration on the intensity signals of the collection of intensity signals based on one or more reference samples. The performing the cross-sample calibration includes: constructing a reference signal distribution based on intensity signals of the one or more reference samples; and for one or more input samples of the set of input samples: obtaining a respective set of intensity signals, of the collection of intensity signals, corresponding to that input sample, the set of intensity signals corresponding to the input sample including (i) a first subset, C, of intensity signals from one or more targeted genomic regions of interest and (ii) a second subset, B, of intensity signals from at least one genomic regions outside the one or more targeted genomic regions of interest, and calibrating the intensity signals in C based on the reference signal distribution, to produce a respective calibrated set of intensity signals corresponding to the input sample. The method additionally includes determining, for the one or more input samples, and from a respective one or more calibrated sets of intensity signals corresponding to the one or more input samples, a respective at least one aggregated calibrated signal from the one or more targeted genomic regions of interest, wherein the determining produces a collection of aggregated calibrated signals, and detecting one or more variants in the one or more targeted genomic regions of interest based on the collection of aggregated calibrated signals.

Yet further, a computer program product including a computer readable storage medium readable by a processing circuit and storing instructions for execution by the processing circuit is provided for performing a method for improved calling of copy number variants in a genomic sequence. The method includes obtaining a collection of intensity signals from assays of a set of input samples including genetic material, and performing a cross-sample calibration on the intensity signals of the collection of intensity signals based on one or more reference samples. The performing the cross-sample calibration includes: constructing a reference signal distribution based on intensity signals of the one or more reference samples; and for one or more input samples of the set of input samples: obtaining a respective set of intensity signals, of the collection of intensity signals, corresponding to that input sample, the set of intensity signals corresponding to the input sample including (i) a first subset, C, of intensity signals from one or more targeted genomic regions of interest and (ii) a second subset, B, of intensity signals from at least one genomic regions outside the one or more targeted genomic regions of interest, and calibrating the intensity signals in C based on the reference signal distribution, to produce a respective calibrated set of intensity signals corresponding to the input sample. The method additionally includes determining, for the one or more input samples, and from a respective one or more calibrated sets of intensity signals corresponding to the one or more input samples, a respective at least one aggregated calibrated signal from the one or more targeted genomic regions of interest, wherein the determining produces a collection of aggregated calibrated signals, and detecting one or more variants in the one or more targeted genomic regions of interest based on the collection of aggregated calibrated signals.

In one or more embodiments, the calibrating of the intensity signals in C, of the set of intensity signals corresponding to the input sample, includes building a mapping for that input sample based on relations between (i) the intensity signals in B and (ii) the reference signal distribution.

In one or more embodiments, the building the mapping includes defining a mapping function M(x) such that M(x) maps intensity signal x as: for x existing in B, M(x)=a matching intensity signal from a vector, A, of reference signal intensities, from the reference signal distribution, corresponding to the at least one genomic regions outside the one or more targeted genomic regions of interest; for x not existing in B but falling between multiple intensity signals in B, M(x)=a linear interpolation based on the M(x) mappings of the multiple intensity signals in B; and for x not existing in B and not falling within a range of the intensity signals in B, M(x)=an extrapolation based on mappings of highest and lowest quantiles in B.

In one or more embodiments, the constructing the reference signal distribution computes the vector A as cross-sample medians of autosomal array probes that are outside the one or more targeted genomic regions of interest.

In one or more embodiments, the calibrating the intensity signals in C further includes using the mapping function to map the intensity signals in C to produce the calibrated set of intensity signals corresponding to the input sample.

In one or more embodiments, the obtaining the collection of intensity signals includes, for the set of input samples, using a set of array hybridization control probes to identify probe hybridization biases by aggregating row-based normalized raw intensity values from the control probes into an aggregated value cₛ, aggregating row-based normalized intensity values from assays targeting human genomic material into an aggregated value xₛ, and determining a contamination factor fₛ as a function of xₛ and cₛ, where fₛ, xₛ and cₛ are determined per input sample.

In one or more embodiments, the function for contamination factor fₛ is: fₛ=xₛ/cₛ.

In one or more embodiments, the determining, for the one or more input samples, and from the respective one or more calibrated sets of intensity signals corresponding to the one or more input samples, the respective at least one aggregated calibrated signal includes, for an aggregated calibrated signal of the at least one aggregated calibrated signal: determining a first aggregated signal from a calibrated set of intensity signals corresponding to a targeted region of the input sample, and using the contamination factor to correct the first aggregated signal and produce a second aggregated signal, wherein the second aggregated signal is output as the aggregated calibrated signal for the targeted region of the input sample.

In one or more embodiments, the using the contamination factor and producing the second aggregated signal includes (i) using a regression-based model to predict contribution of contamination based on the contamination factor, (ii) determining a residue as a function of the first aggregated signal and the contribution of contamination predicted by the model, and (iii) determining the second aggregated signal as a function of the residue and a composite contamination factor from across the input samples.

In one or more embodiments, the one or more variants are one or more copy number variants.

In one or more embodiments, none of (i) deoxyribonucleic acid (DNA) quantification of the input samples, (ii) normalization of the input samples, and (iii) prior measurements of fraction or amount of DNA contaminant in the input samples is known or required in performing the method.

In one or more embodiments, the input samples of the set of input samples contains at least one of (i) variable amounts or concentrations of deoxyribonucleic acid (DNA) relative to each other or (ii) different fractions of contaminant DNA relative to each other.

In one or more embodiments, the collection of intensity signals is from a high-throughput genotyping platform genotyping the input samples using a microarray-based genotyping platform.

Additional features and advantages are realized through the concepts described herein.

### BRIEF DESCRIPTION OF THE DRAWINGS

Aspects described herein are particularly pointed out and distinctly claimed as examples in the claims at the conclusion of the specification. The foregoing and other objects, features, and advantages of the disclosure are apparent from the following detailed description taken in conjunction with the accompanying drawings in which:

FIG. 1 depicts an example approach for constructing a microarray for targeted copy number detection using a high-throughput genotyping platform;

FIGS. 2-3 illustrate example impacts of contamination and total target deoxyribonucleic acid amount on signal intensity;

FIGS. 4-6 illustrate example comparisons between capabilities of aspects described herein and a prior method for CNV calling;

FIG. 7A depicts an example process for array-based targeted copy number variant detection, in accordance with aspects described herein;

FIG. 7B depicts an example process for signal correction based on a contamination factor, in accordance with aspects described herein; and

FIG. 8 depicts an example of a computer system and associated devices to incorporate and/or use aspects described herein.

### DETAILED DESCRIPTION

Described herein are approaches for array-based targeted copy number detection, for instance detection on contaminated and/or variable concentration samples. For instance, example approaches enable, facilitate, and provide accurate copy number determinations on samples, including, for instance, samples with (i) variable amounts of input deoxyribonucleic acid (DNA), and/or (ii) variable fractions of contaminant DNA.

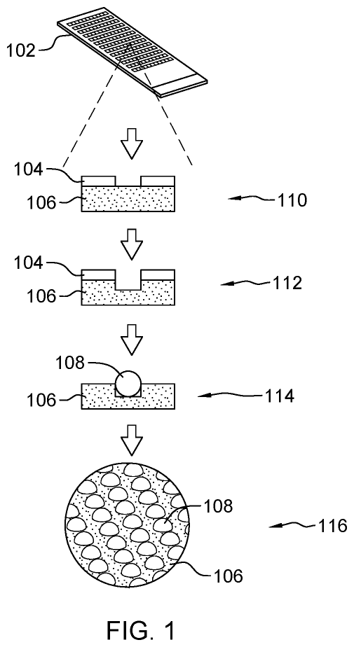

Methods exist for targeted copy number detection using a high-throughput genotyping platform. In one example, a microarray-based genotyping platform is used. FIG. 1 depicts an example approach for constructing a microarray for targeted copy number detection using a high-throughput genotyping platform. Referring to FIG. 1, a silicon wafer 102 is initially obtained and, at 110, a photo resist 104 is disposed/patterned onto substrate layer 106 of the wafer. Plasma etching (112) etches microwells into the substrate 106. This is followed by a cleaning step 114 to remove the photo resist 104. A pattern/array of wells is formed into which beads 108 are disposed to produce (116) the microarray.

The BeadArray microarray technology offered by Illumina, Inc. of San Diego, California is used by way of example. The BeadArray microarray technology uses silica microbeads, in which, on the surface of each array, or BeadChip, hundreds of thousands to millions of genotypes for a single individual can be assayed at once. The tiny silica beads are housed in the carefully etched microwells and coated with multiple copies of an oligonucleotide probe targeting a specific locus in the genome. As DNA fragments pass over the BeadChip, each probe binds to a complementary sequence in the sample DNA, stopping one base before the locus of interest. Allele specificity is conferred by a single base extension that incorporates one of four labeled nucleotides. When excited by a laser, the nucleotide label emits a signal. The intensity of that signal conveys information about the allelic ratio at that locus.

Genetic Variant Detection with Contaminated and/or variable-concentration Saliva DNA Samples: As a non-invasive source for DNA, saliva is an important sample type in genomics. Analysis of saliva DNA enables routine direct-to-consumer (DTC), research, and/or clinical genomics applications. The saliva sample type, however, poses some unique challenges for accurate genetic variant detection due to the presence and variability of non-human contaminant DNA, as well as the variability in total DNA concentration. For instance, it has been shown that false positive rate for SNV detection was slightly higher in saliva and buccal samples, while the sensitivity of CNV detection was up to 25% lower for saliva samples compare to blood, and it has been shown that with whole genome sequencing, over 95% of SNVs found in saliva were concordant with the paired blood samples, while for CNVs only 75% are concordant. In general, CNV detection is much more challenging when dealing with saliva samples as compared to blood samples.

Aspects described herein present methods to address the saliva-specific challenges of CNV detection, and thereby enable more accurate CNV calling for genetic applications, such as pharmacogenomics and carrier screening (as examples) on saliva samples as the DNA source. For instance, aspects enable more accurate saliva DNA-based CNV detection in situations of unknown/variable DNA concentration and/or unknown/variable fraction of contamination. It advantageously does not require DNA quantification or normalization of the input DNA sample, or prior measurements on the fraction or amount of DNA contaminant. Meanwhile, it supports a set of samples each with (i) a different amount/concentration of DNA and/or (ii) a different fraction of contaminant DNA. In accordance with aspects presented herein, CNV detection using a set of saliva samples can work almost as well as detection using a set of normalization DNA samples without contamination.

Signal Aggregation with a Target Genomic Region: To enable CNV detection for a specific target region in the genome, and by way of one specific example, a variable number of target-specific 50-mer DNA probes are designed to provide complementary assays with 3′ DNA ends spanning the target region. The intensity signals from all assays are captured by a scanner (for instance the iScan System offered by Illumina, Inc.), and then individual assays are normalized, weighted, and aggregated.

Specifically, for a target region t for sample s, the aggregated signal xₜₛ is given by Eq. 1 as:

$${x}_{ts} = {\sum }_{i = 1}^{p} {w}_{ti} \cdot {x}_{tis} , \qquad ( Eq . 1 )$$

where i indicates the assay, wₜᵢ is the assay-specific weighting, and xₜᵢₛ is the intensity or normalized intensity signal (e.g., R the total intensity from red and green channel, or LRR the log R ratio) for a specific assay i in sample s.

### A. Cross-Sample Calibration of Intensity Signals Based on Reference Samples

Contamination leads to differences in the intensity profiles among samples with different levels of contaminants and human DNA quality. In a first aspect, an approach is provided to correct for systematic signal differences by (i) constructing a reference signal distribution and (ii) performing reference-based calibration of a sample. A quantile normalization algorithm can operate across a whole chip containing multiple samples.

A reference intensity distribution is constructed by identifying a set of reference samples, where low quality samples have been removed. On the reference set, a process computes a reference intensity vector A, a vector of reference intensity values, as the cross-sample medians of autosomal array probes (e.g., Infinium genotyping assay) that are outside the target regions of interest in the samples. The reference intensity values are further sorted to form A as a reference quantile vector, i.e., as a sorted/ranked list of intensity values. Missing values may be retained as the lowest quantiles during the construction of this reference quantile vector, thus, signal intensities that are low quantile correspond to missing values.

The following process can then be used for calibration of new sample(s). For each new sample, the process splits the array signals for that sample into two subsets: a set B containing intensity signals from all autosomal array probes (e.g., Infinium genotyping assay) that are outside the target region(s) of interest for that sample, and a set C containing all the remaining intensity signals for that sample (i.e., that were not included in set B). Then with A and B, the process defines a mapping M of the signal intensity quantiles, i.e., a function for calibrating any given intensity value x in that sample, as follows:
For any intensity value x in B, M(x) is defined as the matching quantile value in A (as B may be sorted in the same way as to form quantile vectors);
For any intensity value x not in B but falling within the range of intensity values in B (i.e., between two adjacent values in B), M(x) is defined as the linear interpolation based on values in B (for instance an interpolation local to the values in B that are closest to x); and
For any intensity value x outside a range of intensity values in B, M(x) is defined as the extrapolation based on the highest and lowest quantiles (for instance, the top 100 highest intensities and bottom 100 lowest intensities, as an example) in B.

Once the mapping M is defined based on A and B, the process then calibrates every intensity value x in C as M(c) to produce calibrated individual intensities which may be used for target copy number detection. Specifically, for each CNV target t region of sample s, the aggregated signal (xₜₛ) is calculated using the individual calibrated intensities, of C and from that region t, as substituted into Eq. 1 above, i.e., as shown in Eq. 2 as follows:

$${x}_{ts} = {\sum }_{i = 1}^{p} {w}_{ti} \cdot M ( {x}_{tis} ) \qquad ( Eq . 2 )$$

In Eq. 2, the xₜᵢₛ term of Eq. 1 (the ‘regular intensity’) has been replaced with M(xₜᵢₛ), the calibrated intensity. xₜₛ of Eq. 2 thereby provides an aggregated calibrated signal for that region t of sample s, based on which copy number can be determined.

Therefore, the cross-sample calibration of Section A provides, as a first aspect, cross-sample intensity signal calibration based on reference sample intensity signals. In embodiments, this aspect can be used in conjunction with other aspect(s) described herein, for instance aspects described below in Section B (control-based sample-specific contamination adjustment), though such use together is optional. In other words, each aspect (Section A, Section B) could be used separate/independent/apart from the other, if desired.

### B. Internal Control-Based Sample Specific Contamination Adjustment

Control-based sample-specific contamination adjustment is directed to adjustments that address variable levels of contamination in a given sample. This aspect attempts to estimate a contamination level, directly, in the sample, and then adjust an aggregated signal (which may optionally be an aggregated calibrated signal as discussed in Section A above) based on the estimated contamination level.

For each sample, a set of array hybridization control probes (e.g., Infinium genotyping assays) are used to enable the correction of contamination. The controls are used to access the overall probe hybridization biases coming from the experiment itself—e.g., reagent and other assay conditions—rather than from human DNA or non-human DNA contamination. In other words, the controls enable the measurement of the assay efficiency in the DNA's input independently by measuring the assay itself exclusive of the amount of DNA put into the assay. The control intensities are subjected to a normalization procedure, for example a row-wise normalization procedure in accordance with aspects presented herein. Specifically, a process normalizes raw intensities by removing some measure, such as the median, of samples in the same rows on the arrays, and then adding back a global measure (e.g., the global median). The process then aggregates the row-normalized intensities for all hybridization probes into an aggregated value cₛ.

With the same general approach as for the controls, a process aggregates all assays targeting the human autosomes into an aggregated value, xₛ, as a metric for accessing the abundance of the overall human DNA. The x measure therefore provides the intensity from the other (i.e., other than the control) probes, and therefore the ratio of the xₛ and cₛ values is used to represent the proportion of human DNA in the sample. Thus, a Control-adjusted Contamination (CAC) factor is determined as fₛ=xₛ/cₛ.

A process can then use the CAC factor to adjust a target-specific array signal xₜₛ in a sample specific manner before assigning copy numbers. In embodiments in which this contamination adjustment is used in conjunction with the cross-sample calibration approach of Section A above, the xₜₛ being adjusted may be the xₜₛ of Eq. 2 (the aggregated calibrated signal). For instance, a cross-sample calibration on intensity signals based on reference sample(s) is performed in which the reference signal distribution is constructed, intensity signals corresponding to an input sample are obtained and divided into sets B and C, and the intensity signals in C are calibrated as discussed above. Then, after performing this cross-sample calibration, the CAC factor can be used to adjust signal(s), such as individual calibrated signals and/or an aggregated signal xₜₛ. Alternatively, if the control-based sample specific contamination adjustment of Section B is not used conjunction with the cross-sample calibration approach of Section A, then the xₜₛ being adjusted may be the xₜₛ of Eq. 1.

The adjustment of the target-specific array signal xₜₛ using the CAC factor estimates an impact of contamination on the observed signal. Specifically, regression-based model Model, such as a linear or non-linear machine learning model, or any complex prediction model, is built to predict the target intensity signals (xₜₛ) from the CAC metrics fₛ across all samples in order to determine the contribution of contamination on the observed signal (xₜₛ) of the sample. Removal of that contribution from the observed signal provides a “residual”—adjusted intensity signal—as the signal observed and attributable to the copy number, which may be the primary piece of information of interest.

Therefore, the process updates the target intensity signals as the residues of the predictor, i.e., using Eq. 3 as follows:

$${r}_{ts} = {x}_{ts} - Model ( {f}_{s} ) \qquad ( Eq . 3 )$$

An adjusted/corrected intensity signal (x′ₜₛ) is obtained as the residue rₜₛ offset by m, the median of Model (fₛ), over all samples s, i.e.:

$${x}_{ts}^{′} = {r}_{ts} + median \{ Model ( {f}_{⋆} ) \} \qquad ( Eq . 4 )$$

The corrected intensity signals (x′ₜₛ) over the samples are then used to determine copy number. This is in contrast to using the initial intensity signals over the samples (i.e., the xₜₛ of Eq. 1 or Eq. 2).

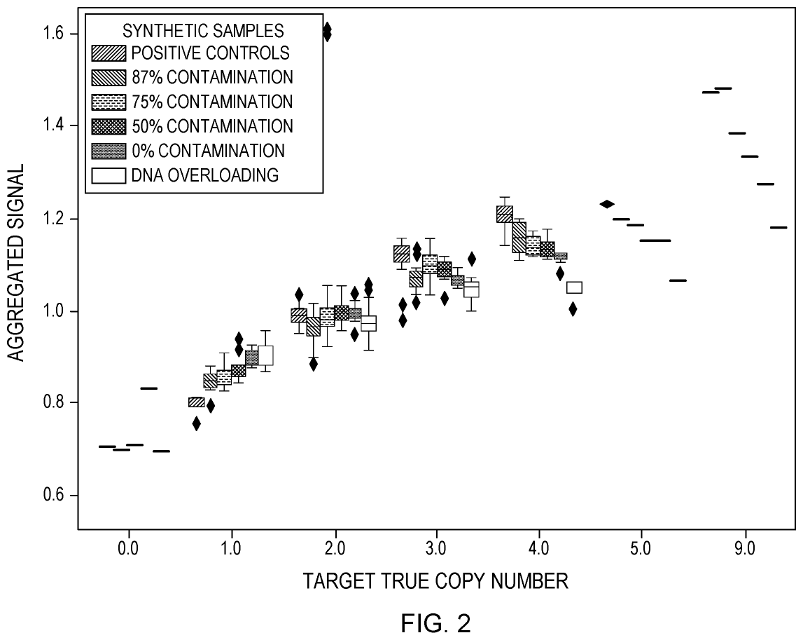

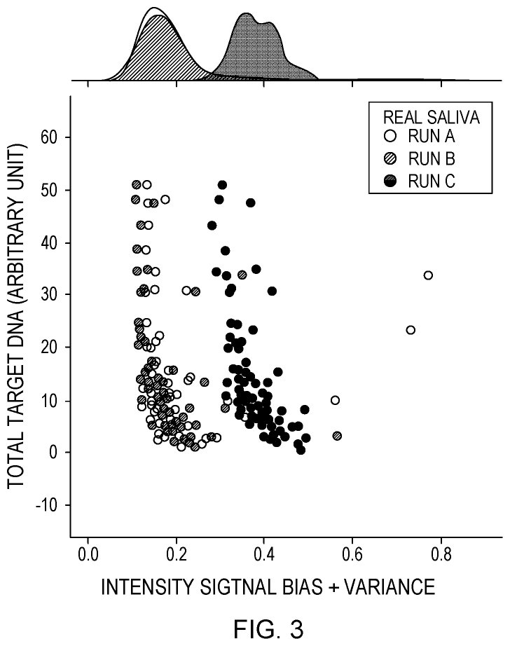

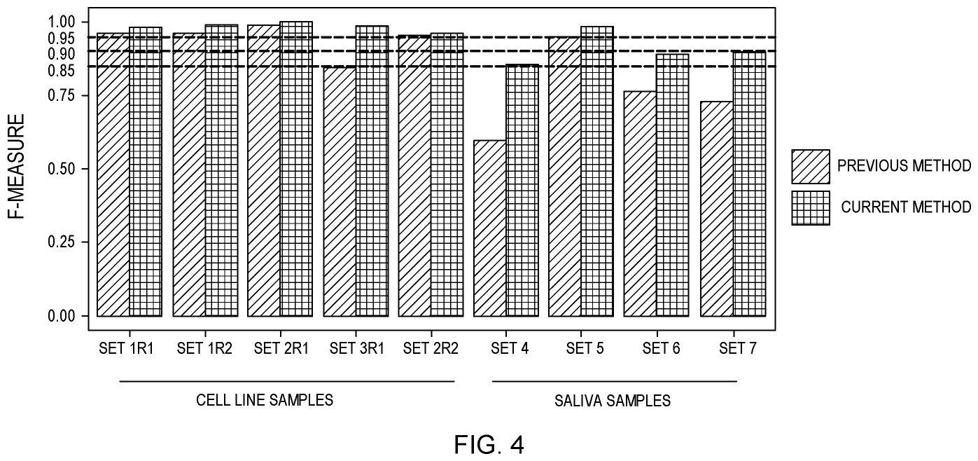

FIGS. 2-3 illustrate example impacts of contamination and total target DNA amount on signal intensity, and FIGS. 4-6 illustrate example comparisons between the capabilities of aspects described herein and a prior method for CNV calling. Specifically, FIG. 2 depicts a graphical representation of the impact of contamination on aggregated intensity signal across target true copy numbers. It is seen that contamination levels in saliva samples directly impact the signal range and signal-to-noise ratio (SNR) coming from the array, and hence adversely affect the copy number assignment. Artificial saliva samples were used in this analysis for demonstrative purposes, and the specific target shown is the CYP2D6 5′ flanking region.

FIG. 3 depicts a graphical representation of the impact of total target DNA input amount on intensity signal variability and biases across three runs. Here the Infinium assay (by way of example) intensity signal variation increases with a decreasing amount of human DNA input. The same set of real saliva samples were run in the three different conditions. In each such condition, a significant correlation was observed between total target DNA amount and signal variability.

FIG. 4 depicts a graphical representation of a comparison between (i) CNV detection methods as disclosed herein (“Current Method”, encompassing both aspects Section A and B above) and (ii) a conventional CNV detection method (“Previous Method”) in terms of CNV Calling accuracy (represented by F-measure) in both (a) cell line DNA samples (i.e., without contamination) and (b) human saliva DNA samples (i.e., with contamination). It is seen that the Current Method provides equal/improved CNV Calling accuracy on (a), and significantly improved CNV calling accuracy on (b). Note that all saliva samples regardless of contamination levels were included in the accuracy evaluation here, however filtering of the saliva sample can lead to further improved accuracy levels.

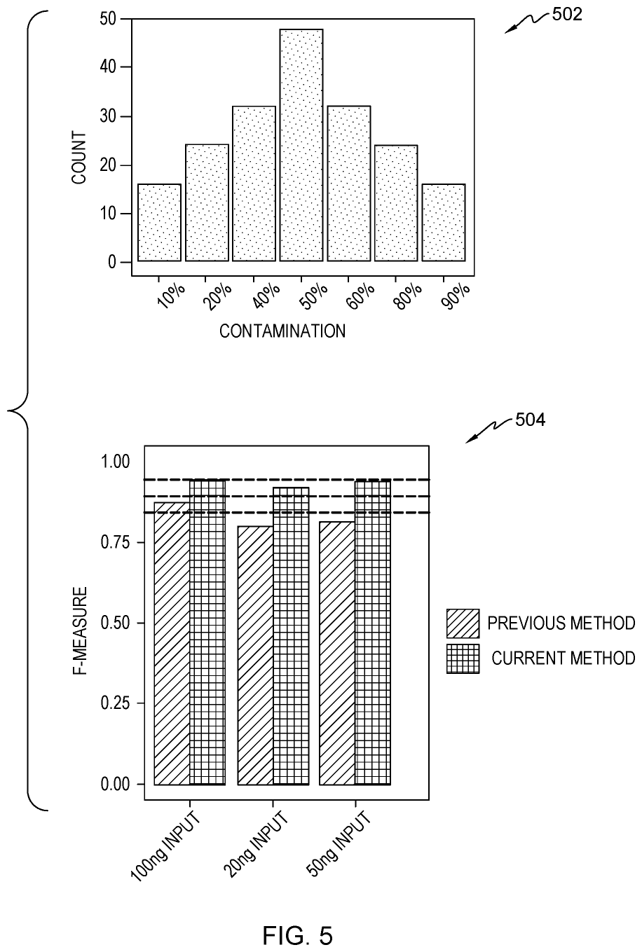

FIG. 5 depicts two graphical representations, 502, 504. First graphical representation (502, on top) provides a representation of artificial contaminated samples created with human DNA mixed with varying degrees of contaminations (ranging from 10% to 90%, centered around 50%). Second graphical representation (504, on bottom) provides a comparison between (i) CNV detection methods as disclosed herein (“Current Method”, encompassing both aspects Section A and B above) and (ii) a conventional CNV detection method (“Previous Method”) in terms of CNV Calling accuracy (represented by F-measure) for the artificial contaminated samples and at three different total DNA input amounts (in nanograms—ng) subjected to array analysis and CNV detection. The proposed CNV detection method disclosed herein (“Current Method”, encompassing both aspects Section A and B above) provided improved accuracy for these artificial samples at all three input amount levels. Note that samples at all contamination levels were included in the accuracy evaluation here, however removal of samples with highest levels of contamination can lead to further improved accuracy levels.

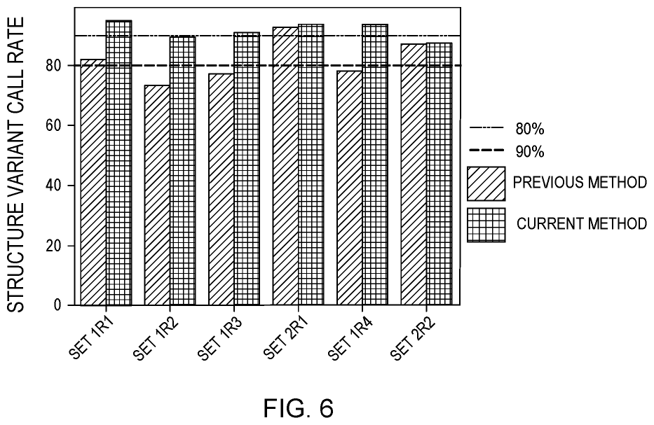

FIG. 6 depicts a graphical representation of a comparison between (i) CNV detection methods as disclosed herein (“Current Method”, encompassing both aspects Section A and B above) and (ii) a conventional CNV detection method (“Previous Method”) in terms of CNV Calling accuracy (represented by F-measure) in structural variant star allele detection for pharmacogenomics gene CYP2D6 across six Sets, and demonstrates improved such detection by the Current Method in comparison to the Previous Method.

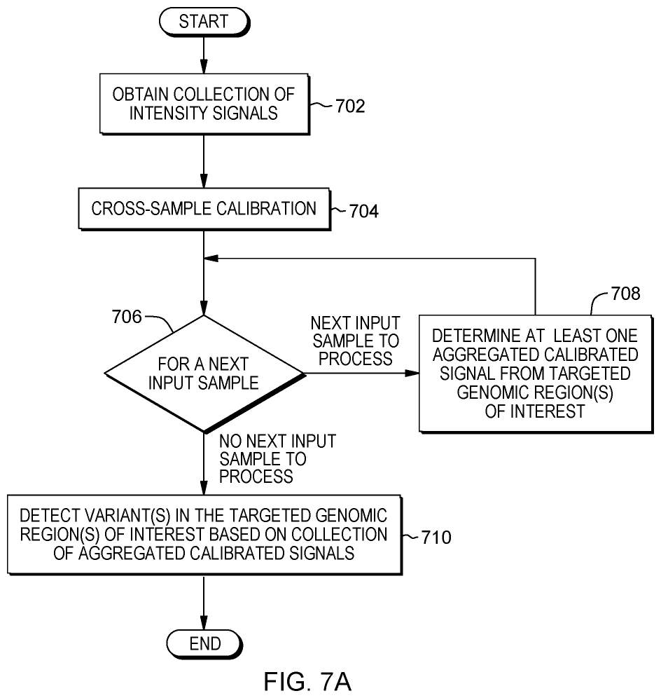

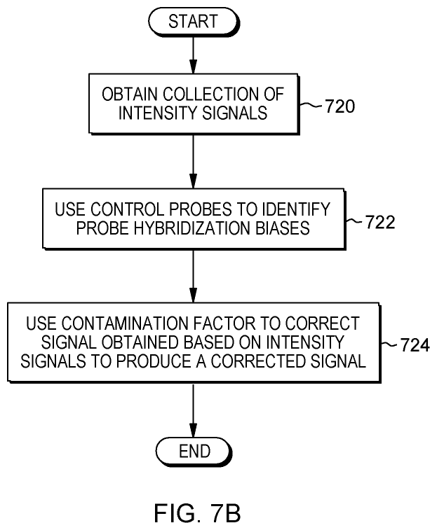

Accordingly, FIGS. 7A and 7B depict example processes in accordance with aspects described herein. The processes may be executed, in one or more examples, by a processor or processing circuitry of one or more computers/computer systems, such as those described herein. For instance, code or instructions implementing the process(es) of FIGS. 7A and/or 7B may be part of modules of software/computer program(s).

FIG. 7A depicts an example process for array-based targeted copy number variant detection. The process may be useful in situations of unknown/variable concentration samples, for example. Referring initially to FIG. 7A, the process obtains (702) a collection of intensity signals from assays of a set of input samples comprising genetic material. In examples, the collection of intensity signals is from a high-throughput genotyping platform genotyping the input samples using a microarray-based genotyping platform. The process continues by performing (704) a cross-sample calibration on the intensity signals of the collection of intensity signals based on reference sample(s).

Performing the cross-sample calibration includes, for example, constructing a reference signal distribution based on intensity signals of the reference sample(s), and also includes, for each of the input sample(s) of the set of input samples, performing (a) obtaining a respective set of intensity signals, of the collection of intensity signals, corresponding to that input sample (where the set of intensity signals corresponding to the input sample includes (i) a first subset, C, of intensity signals from targeted genomic region(s) of interest and (ii) a second subset, B, of intensity signals from genomic region(s) outside the targeted genomic region(s) of interest), and (b) calibrating the intensity signals in C based on the reference signal distribution, to produce a respective calibrated set of intensity signals corresponding to the input sample.

Calibrating of the intensity signals in C, of the set of intensity signals corresponding to the input sample, can include building a mapping for that input sample based on relations between (i) the intensity signals in B and (ii) the reference signal distribution. As an example, building the mapping can include defining a mapping function M(x). By way of example, M(x) can map intensity signal x as: (i) for x existing in B, M(x)=a matching intensity signal from a vector, A, of reference signal intensities, from the reference signal distribution, corresponding to the genomic region(s) outside the targeted genomic region(s) of interest; (ii) for x not existing in B but falling between multiple intensity signals in B, M(x)=a linear interpolation based on the M(x) mappings of the multiple intensity signals in B; and (iii) for x not existing in B and not falling within a range of the intensity signals in B, M(x)=an extrapolation based on mappings of highest and lowest quantiles in B.

In some examples, the constructing the reference signal distribution computes the vector A as cross-sample medians of autosomal array probes that are outside the targeted genomic region(s) of interest.

In some examples, calibrating the intensity signals in C further includes using the mapping function to map the intensity signals in C to produce the calibrated set of intensity signals corresponding to the input sample.

Referring back to FIG. 7A, and based on the cross-sample calibration (704), the process continues by, for each next input sample (706) of the one or more input samples, and from a respective one or more calibrated sets of intensity signals corresponding to the one or more input samples, determining (708) a respective at least one aggregated calibrated signal from the targeted genomic region(s) of interest. The determining can thus produce a collection of aggregated calibrated signals. The process then continues to detecting (710) variant(s) in the targeted genomic region(s) of interest based on the collection of aggregated calibrated signals. In examples, the variant(s) is/are copy number variant(s).

In some examples, obtaining the collection of intensity signals includes correcting for contamination, for instance as described herein above and with reference to FIG. 7B below. For instance, in the context of the process of FIG. 7A, obtaining the collection of intensity signals (702) can include, for the set of input samples, using a set of array hybridization control probes to identify probe hybridization biases by (i) aggregating row-based normalized raw intensity values from the control probes into an aggregated value cₛ, (ii) aggregating row-based normalized intensity values from assays targeting human genomic material into an aggregated value xₛ, and (iii) determining a contamination factor fₛ as a function of xₛ and cₛ, where fₛ, xₛ and cₛ are determined per input sample. In examples, the function for contamination factor fₛ is: fₛ=xₛ/cₛ. Then, in embodiments, the determining (708), for the one or more input samples, and from the respective one or more calibrated sets of intensity signals corresponding to the one or more input samples, the respective at least one aggregated calibrated signal (for instance one per targeted region of the targeted region(s) of the sample) includes, for an aggregated calibrated signal of the at least one aggregated calibrated signal: (i) determining a first aggregated signal from a calibrated set of intensity signals corresponding to a targeted region of the input sample; and using the contamination factor to correct the first aggregated signal and produce a second aggregated signal, where the second aggregated signal is output as the aggregated calibrated signal for the targeted region of the input sample.

In examples, using the contamination factor and producing the second aggregated signal includes using a regression-based model to predict contribution of contamination based on the contamination factor, determining a residue as a function of the first aggregated signal and the contribution of contamination predicted by the model, and determining the second aggregated signal as a function of the residue and a composite contamination factor from across the input samples.

Accordingly, the input samples of the set of input samples can contain at least one of (i) variable amounts or concentrations of DNA relative to each other, or (ii) different fractions of contaminant DNA relative to each other, and, additionally, none of (i) DNA quantification of the input samples, (ii) normalization of the input samples, and (iii) prior measurements of fraction or amount of DNA contaminant in the input samples is known or required, in order to perform processes described herein and arrive at accurate variant detection results.

FIG. 7B depicts an example process for signal correction based on a contamination factor, in accordance with aspects described herein. The signal correction can be performed as part of a process, such as described above with reference to FIG. 7A, or as a standalone correction, as an example. Referring to FIG. 7B, the process obtains (720) a collection of intensity signals from assays of a set of input samples comprising genetic material. The collection of intensity signals is from a high-throughput genotyping platform genotyping the input samples using a microarray-based genotyping platform, for example. The process continues by using (722) a set of array hybridization control probes to identify probe hybridization biases by aggregating row-based normalized raw intensity values from the control probes into an aggregated value cₛ, aggregating row-based normalized intensity values from assays targeting human genomic material into an aggregated value xₛ, and determining a contamination factor fₛ as a function of xₛ and cₛ, where fₛ, xₛ and cₛ are determined per input sample of the set of input samples. In examples, the function for contamination factor fₛ is: fₛ=xₛ/cₛ.

Using the contamination factor, the process corrects (724) a first signal obtained based on intensity signals of the collection of intensity signals and produces a corrected signal. In examples, using the contamination factor and producing the corrected signal includes (i) using a regression-based model to predict contribution of contamination based on the contamination factor, (ii) determining a residue as a function of the first signal and the contribution of contamination predicted by the model, and (iii) determining the corrected signal as a function of the residue and a composite contamination factor from across the input samples. The first signal may be a first aggregated signal from a set of the intensity signals of the collection of intensity signals, with the first aggregated signal corresponding to a target region of an input sample of the set of input samples, and the corrected signal may be a corrected aggregated signal, for instance for that target region of the input sample.

A sampling of aspects described herein is as follows:

A1.A computer-implemented method comprising: obtaining a collection of intensity signals from assays of a set of input samples comprising genetic material; performing a cross-sample calibration on the intensity signals of the collection of intensity signals based on one or more reference samples, the performing the cross-sample calibration comprising: constructing a reference signal distribution based on intensity signals of the one or more reference samples; and for one or more input samples of the set of input samples: obtaining a respective set of intensity signals, of the collection of intensity signals, corresponding to that input sample, the set of intensity signals corresponding to the input sample comprising (i) a first subset, C, of intensity signals from one or more targeted genomic regions of interest and (ii) a second subset, B, of intensity signals from at least one genomic regions outside the one or more targeted genomic regions of interest; and calibrating the intensity signals in C based on the reference signal distribution, to produce a respective calibrated set of intensity signals corresponding to the input sample; determining, for the one or more input samples, and from a respective one or more calibrated sets of intensity signals corresponding to the one or more input samples, a respective at least one aggregated calibrated signal from the one or more targeted genomic regions of interest, wherein the determining produces a collection of aggregated calibrated signals; and detecting one or more variants in the one or more targeted genomic regions of interest based on the collection of aggregated calibrated signals.

A2. The method of A1, wherein the calibrating of the intensity signals in C, of the set of intensity signals corresponding to the input sample, comprises building a mapping for that input sample based on relations between (i) the intensity signals in B and (ii) the reference signal distribution.

A3. The method of A2, wherein the building the mapping comprises defining a mapping function M(x) such that M(x) maps intensity signal x as: for x existing in B, M(x)=a matching intensity signal from a vector, A, of reference signal intensities, from the reference signal distribution, corresponding to the at least one genomic regions outside the one or more targeted genomic regions of interest; for x not existing in B but falling between multiple intensity signals in B, M(x)=a linear interpolation based on the M(x) mappings of the multiple intensity signals in B; and for x not existing in B and not falling within a range of the intensity signals in B, M(x)=an extrapolation based on mappings of highest and lowest quantiles in B.

A4. The method of A3, wherein the constructing the reference signal distribution computes the vector A as cross-sample medians of autosomal array probes that are outside the one or more targeted genomic regions of interest.

A5. The method of A3 or A4, wherein the calibrating the intensity signals in C further comprises using the mapping function to map the intensity signals in C to produce the calibrated set of intensity signals corresponding to the input sample.

A6. The method of A1, A2, A3, A4, or A5, wherein the obtaining the collection of intensity signals comprises, for the set of input samples, using a set of array hybridization control probes to identify probe hybridization biases by aggregating row-based normalized raw intensity values from the control probes into an aggregated value cₛ, aggregating row-based normalized intensity values from assays targeting human genomic material into an aggregated value xₛ, and determining a contamination factor fₛ as a function of xₛ and cₛ, where fₛ, xₛ and cₛ are determined per input sample.

A7. The method of A6, wherein the function for contamination factor fₛ is:

$${f}_{s} = {x}_{s} / {c}_{s} .$$

A8. The method of A6 or A7, wherein the determining, for the one or more input samples, and from the respective one or more calibrated sets of intensity signals corresponding to the one or more input samples, the respective at least one aggregated calibrated signal comprises, for an aggregated calibrated signal of the at least one aggregated calibrated signal: determining a first aggregated signal from a calibrated set of intensity signals corresponding to a targeted region of the input sample; and using the contamination factor to correct the first aggregated signal and produce a second aggregated signal, wherein the second aggregated signal is output as the aggregated calibrated signal for the targeted region of the input sample.

A9. The method of A8, wherein the using the contamination factor and producing the second aggregated signal comprises (i) using a regression-based model to predict contribution of contamination based on the contamination factor, (ii) determining a residue as a function of the first aggregated signal and the contribution of contamination predicted by the model, and (iii) determining the second aggregated signal as a function of the residue and a composite contamination factor from across the input samples.

A10. The method of A1, A2, A3, A4, A5, A6, A7, A8, or A9, wherein the one or more variants are one or more copy number variants.

A11. The method of A1, A2, A3, A4, A5, A6, A7, A8, A9, or A10, wherein none of (i) deoxyribonucleic acid (DNA) quantification of the input samples, (ii) normalization of the input samples, and (iii) prior measurements of fraction or amount of DNA contaminant in the input samples is known or required in performing the method.

A12. The method of A1, A2, A3, A4, A5, A6, A7, A8, A9, A10, or A11, wherein the input samples of the set of input samples contains at least one of (i) variable amounts or concentrations of deoxyribonucleic acid (DNA) relative to each other or (ii) different fractions of contaminant DNA relative to each other.

A13. The method of A1, A2, A3, A4, A5, A6, A7, A8, A9, A10, A11, or A12, wherein the collection of intensity signals is from a high-throughput genotyping platform genotyping the input samples using a microarray-based genotyping platform.

B1.A computer system comprising: a memory; and a processor in communication with the memory, wherein the computer system is configured to perform a method comprising: obtaining a collection of intensity signals from assays of a set of input samples comprising genetic material; performing a cross-sample calibration on the intensity signals of the collection of intensity signals based on one or more reference samples, the performing the cross-sample calibration comprising: constructing a reference signal distribution based on intensity signals of the one or more reference samples; and for one or more input samples of the set of input samples: obtaining a respective set of intensity signals, of the collection of intensity signals, corresponding to that input sample, the set of intensity signals corresponding to the input sample comprising (i) a first subset, C, of intensity signals from one or more targeted genomic regions of interest and (ii) a second subset, B, of intensity signals from at least one genomic regions outside the one or more targeted genomic regions of interest; and calibrating the intensity signals in C based on the reference signal distribution, to produce a respective calibrated set of intensity signals corresponding to the input sample; determining, for the one or more input samples, and from a respective one or more calibrated sets of intensity signals corresponding to the one or more input samples, a respective at least one aggregated calibrated signal from the one or more targeted genomic regions of interest, wherein the determining produces a collection of aggregated calibrated signals; and detecting one or more variants in the one or more targeted genomic regions of interest based on the collection of aggregated calibrated signals.

B2. The computer system of B1, wherein the calibrating of the intensity signals in C, of the set of intensity signals corresponding to the input sample, comprises building a mapping for that input sample based on relations between (i) the intensity signals in B and (ii) the reference signal distribution.

B3. The computer system of B2, wherein the building the mapping comprises defining a mapping function M(x) such that M(x) maps intensity signal x as: for x existing in B, M(x)=a matching intensity signal from a vector, A, of reference signal intensities, from the reference signal distribution, corresponding to the at least one genomic regions outside the one or more targeted genomic regions of interest; for x not existing in B but falling between multiple intensity signals in B, M(x)=a linear interpolation based on the M(x) mappings of the multiple intensity signals in B; and for x not existing in B and not falling within a range of the intensity signals in B, M(x)=an extrapolation based on mappings of highest and lowest quantiles in B.

B4. The computer system of B3, wherein the constructing the reference signal distribution computes the vector A as cross-sample medians of autosomal array probes that are outside the one or more targeted genomic regions of interest.

B5. The computer system of B3 or B4, wherein the calibrating the intensity signals in C further comprises using the mapping function to map the intensity signals in C to produce the calibrated set of intensity signals corresponding to the input sample.

B6. The computer system of B1, B2, B3, B4, or B5, wherein the obtaining the collection of intensity signals comprises, for the set of input samples, using a set of array hybridization control probes to identify probe hybridization biases by aggregating row-based normalized raw intensity values from the control probes into an aggregated value cₛ, aggregating row-based normalized intensity values from assays targeting human genomic material into an aggregated value xₛ, and determining a contamination factor fₛ as a function of xₛ and cₛ, where fₛ, xₛ and cₛ are determined per input sample.

B7. The computer system of B6, wherein the function for contamination factor fₛ is: fₛ=xₛ/cₛ.

B8. The computer system of B6 or B7, wherein the determining, for the one or more input samples, and from the respective one or more calibrated sets of intensity signals corresponding to the one or more input samples, the respective at least one aggregated calibrated signal comprises, for an aggregated calibrated signal of the at least one aggregated calibrated signal: determining a first aggregated signal from a calibrated set of intensity signals corresponding to a targeted region of the input sample; and using the contamination factor to correct the first aggregated signal and produce a second aggregated signal, wherein the second aggregated signal is output as the aggregated calibrated signal for the targeted region of the input sample.

B9. The computer system of B8, wherein the using the contamination factor and producing the second aggregated signal comprises (i) using a regression-based model to predict contribution of contamination based on the contamination factor, (ii) determining a residue as a function of the first aggregated signal and the contribution of contamination predicted by the model, and (iii) determining the second aggregated signal as a function of the residue and a composite contamination factor from across the input samples.

B10. The computer system of B1, B2, B3, B4, B5, B6, B7, B8, or B9, wherein the one or more variants are one or more copy number variants.

B11. The computer system of B1, B2, B3, B4, B5, B6, B7, B8, B9, or B10, wherein none of (i) deoxyribonucleic acid (DNA) quantification of the input samples, (ii) normalization of the input samples, and (iii) prior measurements of fraction or amount of DNA contaminant in the input samples is known or required in performing the method.

B12. The computer system of B1, B2, B3, B4, B5, B6, B7, B8, B9, B10, or B11, wherein the input samples of the set of input samples contains at least one of (i) variable amounts or concentrations of deoxyribonucleic acid (DNA) relative to each other or (ii) different fractions of contaminant DNA relative to each other.

B13. The computer system of B1, B2, B3, B4, B5, B6, B7, B8, B9, B10, B11, or B12, wherein the collection of intensity signals is from a high-throughput genotyping platform genotyping the input samples using a microarray-based genotyping platform.

C1. A computer program product comprising: a computer readable storage medium readable by a processing circuit and storing instructions for execution by the processing circuit for performing a method comprising: obtaining a collection of intensity signals from assays of a set of input samples comprising genetic material; performing a cross-sample calibration on the intensity signals of the collection of intensity signals based on one or more reference samples, the performing the cross-sample calibration comprising: constructing a reference signal distribution based on intensity signals of the one or more reference samples; and for one or more input samples of the set of input samples: obtaining a respective set of intensity signals, of the collection of intensity signals, corresponding to that input sample, the set of intensity signals corresponding to the input sample comprising (i) a first subset, C, of intensity signals from one or more targeted genomic regions of interest and (ii) a second subset, B, of intensity signals from at least one genomic regions outside the one or more targeted genomic regions of interest; and calibrating the intensity signals in C based on the reference signal distribution, to produce a respective calibrated set of intensity signals corresponding to the input sample; determining, for the one or more input samples, and from a respective one or more calibrated sets of intensity signals corresponding to the one or more input samples, a respective at least one aggregated calibrated signal from the one or more targeted genomic regions of interest, wherein the determining produces a collection of aggregated calibrated signals; and detecting one or more variants in the one or more targeted genomic regions of interest based on the collection of aggregated calibrated signals.

C2. The computer program product of C1, wherein the calibrating of the intensity signals in C, of the set of intensity signals corresponding to the input sample, comprises building a mapping for that input sample based on relations between (i) the intensity signals in B and (ii) the reference signal distribution.

C3. The computer program product of C2, wherein the building the mapping comprises defining a mapping function M(x) such that M(x) maps intensity signal x as: for x existing in B, M(x)=a matching intensity signal from a vector, A, of reference signal intensities, from the reference signal distribution, corresponding to the at least one genomic regions outside the one or more targeted genomic regions of interest; for x not existing in B but falling between multiple intensity signals in B, M(x)=a linear interpolation based on the M(x) mappings of the multiple intensity signals in B; and for x not existing in B and not falling within a range of the intensity signals in B, M(x)=an extrapolation based on mappings of highest and lowest quantiles in B.

C4. The computer program product of C3, wherein the constructing the reference signal distribution computes the vector A as cross-sample medians of autosomal array probes that are outside the one or more targeted genomic regions of interest.

C5. The computer program product of C3 or C4, wherein the calibrating the intensity signals in C further comprises using the mapping function to map the intensity signals in C to produce the calibrated set of intensity signals corresponding to the input sample.

C6. The computer program product of C1, C2, C3, C4, or C5, wherein the obtaining the collection of intensity signals comprises, for the set of input samples, using a set of array hybridization control probes to identify probe hybridization biases by aggregating row-based normalized raw intensity values from the control probes into an aggregated value cₛ, aggregating row-based normalized intensity values from assays targeting human genomic material into an aggregated value xₛ, and determining a contamination factor fₛ as a function of xₛ and cₛ, where fₛ, xₛ and cₛ are determined per input sample.

C7. The computer program product of C6, wherein the function for contamination factor fₛ is: fₛ=xₛ/cₛ.

C8. The computer program product of C6 or C7, wherein the determining, for the one or more input samples, and from the respective one or more calibrated sets of intensity signals corresponding to the one or more input samples, the respective at least one aggregated calibrated signal comprises, for an aggregated calibrated signal of the at least one aggregated calibrated signal: determining a first aggregated signal from a calibrated set of intensity signals corresponding to a targeted region of the input sample; and using the contamination factor to correct the first aggregated signal and produce a second aggregated signal, wherein the second aggregated signal is output as the aggregated calibrated signal for the targeted region of the input sample.

C9. The computer program product of C8, wherein the using the contamination factor and producing the second aggregated signal comprises (i) using a regression-based model to predict contribution of contamination based on the contamination factor, (ii) determining a residue as a function of the first aggregated signal and the contribution of contamination predicted by the model, and (iii) determining the second aggregated signal as a function of the residue and a composite contamination factor from across the input samples.

C10. The computer program product of C1, C2, C3, C4, C5, C6, C7, C8, or C9, wherein the one or more variants are one or more copy number variants.

C11. The computer program product of C1, C2, C3, C4, C5, C6, C7, C8, C9, or C10, wherein none of (i) deoxyribonucleic acid (DNA) quantification of the input samples, (ii) normalization of the input samples, and (iii) prior measurements of fraction or amount of DNA contaminant in the input samples is known or required in performing the method.

C12. The computer program product of C1, C2, C3, C4, C5, C6, C7, C8, C9, C10, or C11, wherein the input samples of the set of input samples contains at least one of (i) variable amounts or concentrations of deoxyribonucleic acid (DNA) relative to each other or (ii) different fractions of contaminant DNA relative to each other.

C13. The computer program product of C1, C2, C3, C4, C5, C6, C7, C8, C9, C10, C11, or C12, wherein the collection of intensity signals is from a high-throughput genotyping platform genotyping the input samples using a microarray-based genotyping platform.

D1.A computer-implemented method comprising: obtaining a collection of intensity signals from assays of a set of input samples comprising genetic material; using a set of array hybridization control probes to identify probe hybridization biases by aggregating row-based normalized raw intensity values from the control probes into an aggregated value cₛ, aggregating row-based normalized intensity values from assays targeting human genomic material into an aggregated value xₛ, and determining a contamination factor fₛ as a function of xₛ and cₛ, where fₛ, xₛ and cₛ are determined per input sample of the set of input samples; and using the contamination factor to correct a first signal obtained based on intensity signals of the collection of intensity signals and produce a corrected signal.

D2. The method of D1, wherein using the contamination factor and producing the corrected signal comprises (i) using a regression-based model to predict contribution of contamination based on the contamination factor, (ii) determining a residue as a function of the first signal and the contribution of contamination predicted by the model, and (iii) determining the corrected signal as a function of the residue and a composite contamination factor from across the input samples.

D3. The method of D1 or D2, wherein the function for contamination factor fₛ is: fₛ=xₛ/cₛ.

D4. The method of D1, D2, or D3, wherein the first signal is a first aggregated signal from a set of the intensity signals of the collection of intensity signals, the first aggregated signal corresponding to a target region of an input sample of the set of input samples, and wherein the corrected signal is a corrected aggregated signal.

D5. The method of D1, D2, D3, or D4, wherein the collection of intensity signals is from a high-throughput genotyping platform genotyping the input samples using a microarray-based genotyping platform.

E1. A computer system comprising: a memory; and a processor in communication with the memory, wherein the computer system is configured to perform a method comprising: obtaining a collection of intensity signals from assays of a set of input samples comprising genetic material; using a set of array hybridization control probes to identify probe hybridization biases by aggregating row-based normalized raw intensity values from the control probes into an aggregated value cₛ, aggregating row-based normalized intensity values from assays targeting human genomic material into an aggregated value xₛ, and determining a contamination factor fₛ as a function of xₛ and cₛ, where fₛ, xₛ and cₛ are determined per input sample of the set of input samples; and using the contamination factor to correct a first signal obtained based on intensity signals of the collection of intensity signals and produce a corrected signal.

E2. The computer system of E1, wherein using the contamination factor and producing the corrected signal comprises (i) using a regression-based model to predict contribution of contamination based on the contamination factor, (ii) determining a residue as a function of the first signal and the contribution of contamination predicted by the model, and (iii) determining the corrected signal as a function of the residue and a composite contamination factor from across the input samples.

E3. The computer system of E1 or E2, wherein the function for contamination factor fₛ is: fₛ=xₛ/cₛ.

E4. The computer system of E1, E2, or E3, wherein the first signal is a first aggregated signal from a set of the intensity signals of the collection of intensity signals, the first aggregated signal corresponding to a target region of an input sample of the set of input samples, and wherein the corrected signal is a corrected aggregated signal.

E5. The computer system of E1, E2, E3, or E4, wherein the collection of intensity signals is from a high-throughput genotyping platform genotyping the input samples using a microarray-based genotyping platform.

F1. A computer program product comprising: a computer readable storage medium readable by a processing circuit and storing instructions for execution by the processing circuit for performing a method comprising: obtaining a collection of intensity signals from assays of a set of input samples comprising genetic material; using a set of array hybridization control probes to identify probe hybridization biases by aggregating row-based normalized raw intensity values from the control probes into an aggregated value cₛ, aggregating row-based normalized intensity values from assays targeting human genomic material into an aggregated value xₛ, and determining a contamination factor fₛ as a function of xₛ and cₛ, where fₛ, xₛ and cₛ are determined per input sample of the set of input samples; and using the contamination factor to correct a first signal obtained based on intensity signals of the collection of intensity signals and produce a corrected signal.

F2. The computer program product of F1, wherein using the contamination factor and producing the corrected signal comprises (i) using a regression-based model to predict contribution of contamination based on the contamination factor, (ii) determining a residue as a function of the first signal and the contribution of contamination predicted by the model, and (iii) determining the corrected signal as a function of the residue and a composite contamination factor from across the input samples.

F3. The computer program product of F1 or F2, wherein the function for contamination factor fₛ is: fₛ=xₛ/cₛ.

F4. The computer program product of F1, F2, or F3, wherein the first signal is a first aggregated signal from a set of the intensity signals of the collection of intensity signals, the first aggregated signal corresponding to a target region of an input sample of the set of input samples, and wherein the corrected signal is a corrected aggregated signal.

F5. The computer program product of F1, F2, F3, or F4, wherein the collection of intensity signals is from a high-throughput genotyping platform genotyping the input samples using a microarray-based genotyping platform.

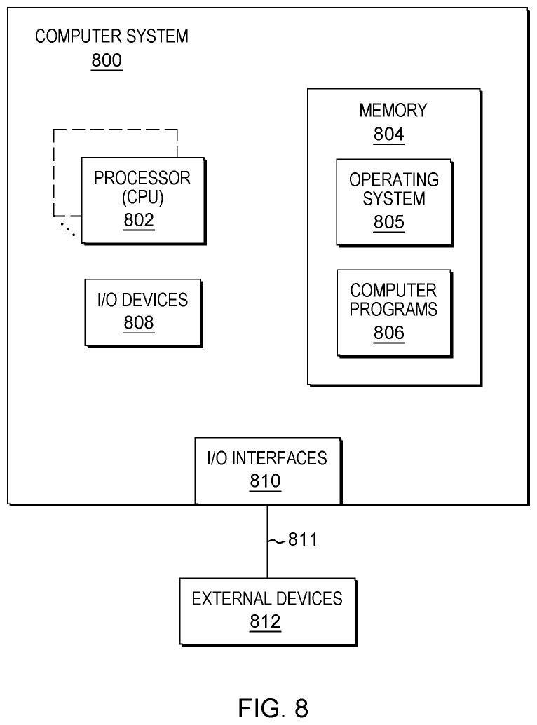

Processes described herein may be performed singly or collectively by one or more computer systems, such as one or more computer system(s) executing genomic analysis software to perform aspects described herein. FIG. 8 depicts an example of a computer system and associated devices to incorporate and/or use aspects described herein. A computer system may also be referred to herein as a data processing device/system, computing device/system/node, or simply a computer. The computer system may be based on one or more of various system architectures and/or instruction set architectures, such as those offered by Intel Corporation (Santa Clara, California, USA) as an example. FIG. 8 shows a computer system 800 in communication with external device(s) 812. Computer system 800 includes one or more processor(s) 802, for instance central processing unit(s) (CPUs). A processor can include functional components used in the execution of instructions, such as functional components to fetch program instructions from locations such as cache or main memory, decode program instructions, and execute program instructions, access memory for instruction execution, and write results of the executed instructions. A processor 802 can also include register(s) to be used by one or more of the functional components. Computer system 800 also includes memory 804, input/output (I/O) devices 808, and I/O interfaces 810, which may be coupled to processor(s) 802 and each other via one or more buses and/or other connections. Bus connections represent one or more of any of several types of bus structures, including a memory bus or memory controller, a peripheral bus, an accelerated graphics port, and a processor or local bus using any of a variety of bus architectures. By way of example, and not limitation, such architectures include the Industry Standard Architecture (ISA), the Micro Channel Architecture (MCA), the Enhanced ISA (EISA), the Video Electronics Standards Association (VESA) local bus, and the Peripheral Component Interconnect (PCI).

Memory 804 can be or include main or system memory (e.g. Random Access Memory) used in the execution of program instructions, storage device(s) such as hard drive(s), flash media, or optical media as examples, and/or cache memory, as examples. Memory 804 can include, for instance, a cache, such as a shared cache, which may be coupled to local caches (examples include L1 cache, L2 cache, etc.) of processor(s) 802. Additionally, memory 804 may be or include at least one computer program product having a set (e.g., at least one) of program modules, instructions, code or the like that is/are configured to carry out functions of embodiments described herein when executed by one or more processors.

Memory 804 can store an operating system 805 and other computer programs 806, such as one or more computer programs/applications that execute to perform aspects described herein. Specifically, programs/applications can include computer readable program instructions that may be configured to carry out functions of embodiments of aspects described herein.

Examples of I/O devices 808 include but are not limited to microphones, speakers, Global Positioning System (GPS) devices, cameras, lights, accelerometers, gyroscopes, magnetometers, sensor devices configured to sense light, proximity, heart rate, body and/or ambient temperature, blood pressure, and/or skin resistance, and activity monitors. An I/O device may be incorporated into the computer system as shown, though in some embodiments an I/O device may be regarded as an external device (812) coupled to the computer system through one or more I/O interfaces 810.

Computer system 800 may communicate with one or more external devices 812 via one or more I/O interfaces 810. Example external devices include a keyboard, a pointing device, a display, and/or any other devices that enable a user to interact with computer system 800. Other example external devices include any device that enables computer system 800 to communicate with one or more other computing systems or peripheral devices such as a printer. A network interface/adapter is an example I/O interface that enables computer system 800 to communicate with one or more networks, such as a local area network (LAN), a general wide area network (WAN), and/or a public network (e.g., the Internet), providing communication with other computing devices or systems, storage devices, or the like. Ethernet-based (such as Wi-Fi) interfaces and Bluetooth® adapters are just examples of the currently available types of network adapters used in computer systems (BLUETOOTH is a registered trademark of Bluetooth SIG, Inc., Kirkland, Washington, U.S.A.).

The communication between I/O interfaces 810 and external devices 812 can occur across wired and/or wireless communications link(s) 811, such as Ethernet-based wired or wireless connections. Example wireless connections include cellular, Wi-Fi, Bluetooth®, proximity-based, near-field, or other types of wireless connections. More generally, communications link(s) 811 may be any appropriate wireless and/or wired communication link(s) for communicating data.

Particular external device(s) 812 may include one or more data storage devices, which may store one or more programs, one or more computer readable program instructions, and/or data, etc. Computer system 800 may include and/or be coupled to and in communication with (e.g. as an external device of the computer system) removable/non-removable, volatile/non-volatile computer system storage media. For example, it may include and/or be coupled to a non-removable, non-volatile magnetic media (typically called a “hard drive”), a magnetic disk drive for reading from and writing to a removable, non-volatile magnetic disk (e.g., a “floppy disk”), and/or an optical disk drive for reading from or writing to a removable, non-volatile optical disk, such as a CD-ROM, DVD-ROM or other optical media.

Computer system 800 may be operational with numerous other general purpose or special purpose computing system environments or configurations. Computer system 800 may take any of various forms, well-known examples of which include, but are not limited to, personal computer (PC) system(s), server computer system(s), such as messaging server(s), thin client(s), thick client(s), workstation(s), laptop(s), handheld device(s), mobile device(s)/computer(s) such as smartphone(s), tablet(s), and wearable device(s), multiprocessor system(s), microprocessor-based system(s), telephony device(s), network appliance(s) (such as edge appliance(s)), virtualization device(s), storage controller(s), set top box(es), programmable consumer electronic(s), network PC(s), minicomputer system(s), mainframe computer system(s), and distributed cloud computing environment(s) that include any of the above systems or devices, and the like.

Aspects of the present invention may be a system, a method, and/or a computer program product, any of which may be configured to perform or facilitate aspects described herein.

In some embodiments, aspects of the present invention may take the form of a computer program product, which may be embodied as computer readable medium(s). A computer readable medium may be a tangible storage device/medium having computer readable program code/instructions stored thereon. Example computer readable medium(s) include, but are not limited to, electronic, magnetic, optical, or semiconductor storage devices or systems, or any combination of the foregoing. Example embodiments of a computer readable medium include a hard drive or other mass-storage device, an electrical connection having wires, random access memory (RAM), read-only memory (ROM), erasable-programmable read-only memory such as EPROM or flash memory, an optical fiber, a portable computer disk/diskette, such as a compact disc read-only memory (CD-ROM) or Digital Versatile Disc (DVD), an optical storage device, a magnetic storage device, or any combination of the foregoing. The computer readable medium may be readable by a processor, processing unit, or the like, to obtain data (e.g. instructions) from the medium for execution. In a particular example, a computer program product is or includes one or more computer readable media that includes/stores computer readable program code to provide and facilitate one or more aspects described herein.

As noted, program instruction contained or stored in/on a computer readable medium can be obtained and executed by any of various suitable components such as a processor of a computer system to cause the computer system to behave and function in a particular manner. Such program instructions for carrying out operations to perform, achieve, or facilitate aspects described herein may be written in, or compiled from code written in, any desired programming language. In some embodiments, such programming language includes object-oriented and/or procedural programming languages such as C, C++, C #, Java, etc.

Program code can include one or more program instructions obtained for execution by one or more processors. Computer program instructions may be provided to one or more processors of, e.g., one or more computer systems, to produce a machine, such that the program instructions, when executed by the one or more processors, perform, achieve, or facilitate aspects of the present invention, such as actions or functions described in flowcharts and/or block diagrams described herein. Thus, each block, or combinations of blocks, of the flowchart illustrations and/or block diagrams depicted and described herein can be implemented, in some embodiments, by computer program instructions.

Although various embodiments are described above, these are only examples.

The terminology used herein is for the purpose of describing particular embodiments only and is not intended to be limiting. As used herein, the singular forms “a”, “an” and “the” are intended to include the plural forms as well, unless the context clearly indicates otherwise. It will be further understood that the terms “comprises” and/or “comprising”, when used in this specification, specify the presence of stated features, integers, steps, operations, elements, and/or components, but do not preclude the presence or addition of one or more other features, integers, steps, operations, elements, components and/or groups thereof.

The corresponding structures, materials, acts, and equivalents of all means or step plus function elements in the claims below, if any, are intended to include any structure, material, or act for performing the function in combination with other claimed elements as specifically claimed. The description of one or more embodiments has been presented for purposes of illustration and description, but is not intended to be exhaustive or limited to in the form disclosed. Many modifications and variations will be apparent to those of ordinary skill in the art. The embodiment was chosen and described in order to best explain various aspects and the practical application, and to enable others of ordinary skill in the art to understand various embodiments with various modifications as are suited to the particular use contemplated.

What is claimed is:

1. A computer-implemented method comprising:
obtaining a collection of intensity signals from assays of a set of input samples comprising genetic material;
performing a cross-sample calibration on the intensity signals of the collection of intensity signals based on one or more reference samples, the performing the cross-sample calibration comprising:
constructing a reference signal distribution based on intensity signals of the one or more reference samples; and
for one or more input samples of the set of input samples:
obtaining a respective set of intensity signals, of the collection of intensity signals, corresponding to that input sample, the set of intensity signals corresponding to the input sample comprising (i) a first subset, C, of intensity signals from one or more targeted genomic regions of interest and (ii) a second subset, B, of intensity signals from at least one genomic regions outside the one or more targeted genomic regions of interest; and
calibrating the intensity signals in C based on the reference signal distribution, to produce a respective calibrated set of intensity signals corresponding to the input sample;
determining, for the one or more input samples, and from a respective one or more calibrated sets of intensity signals corresponding to the one or more input samples, a respective at least one aggregated calibrated signal from the one or more targeted genomic regions of interest, wherein the determining produces a collection of aggregated calibrated signals; and
detecting one or more variants in the one or more targeted genomic regions of interest based on the collection of aggregated calibrated signals.

2. The method of claim 1, wherein the calibrating of the intensity signals in C, of the set of intensity signals corresponding to the input sample, comprises building a mapping for that input sample based on relations between (i) the intensity signals in B and (ii) the reference signal distribution.

3. The method of claim 2, wherein the building the mapping comprises defining a mapping function M(x) such that M(x) maps intensity signal x as:
for x existing in B, M(x)=a matching intensity signal from a vector, A, of reference signal intensities, from the reference signal distribution, corresponding to the at least one genomic regions outside the one or more targeted genomic regions of interest;
for x not existing in B but falling between multiple intensity signals in B, M(x)=a linear interpolation based on the M(x) mappings of the multiple intensity signals in B; and
for x not existing in B and not falling within a range of the intensity signals in B, M(x)=an extrapolation based on mappings of highest and lowest quantiles in B.

4. The method of claim 3, wherein the constructing the reference signal distribution computes the vector A as cross-sample medians of autosomal array probes that are outside the one or more targeted genomic regions of interest.

5. The method of claim 3, wherein the calibrating the intensity signals in C further comprises using the mapping function to map the intensity signals in C to produce the calibrated set of intensity signals corresponding to the input sample.

6. The method of claim 1, wherein the obtaining the collection of intensity signals comprises, for the set of input samples, using a set of array hybridization control probes to identify probe hybridization biases by aggregating row-based normalized raw intensity values from the control probes into an aggregated value cₛ, aggregating row-based normalized intensity values from assays targeting human genomic material into an aggregated value xₛ, and determining a contamination factor fₛ as a function of xₛ and cₛ, where fₛ, xₛ and cₛ are determined per input sample.

7. The method of claim 6, wherein the function for contamination factor fₛ is:
$${f}_{s} = {x}_{s} / {c}_{s} .$$

8. The method of claim 6, wherein the determining, for the one or more input samples, and from the respective one or more calibrated sets of intensity signals corresponding to the one or more input samples, the respective at least one aggregated calibrated signal comprises, for an aggregated calibrated signal of the at least one aggregated calibrated signal:
determining a first aggregated signal from a calibrated set of intensity signals corresponding to a targeted region of the input sample; and
using the contamination factor to correct the first aggregated signal and produce a second aggregated signal, wherein the second aggregated signal is output as the aggregated calibrated signal for the targeted region of the input sample.

9. The method of claim 8, wherein the using the contamination factor and producing the second aggregated signal comprises (i) using a regression-based model to predict contribution of contamination based on the contamination factor, (ii) determining a residue as a function of the first aggregated signal and the contribution of contamination predicted by the model, and (iii) determining the second aggregated signal as a function of the residue and a composite contamination factor from across the input samples.

10. The method of claim 1, wherein the one or more variants are one or more copy number variants.

11. The method of claim 1, wherein none of (i) deoxyribonucleic acid (DNA) quantification of the input samples, (ii) normalization of the input samples, and (iii) prior measurements of fraction or amount of DNA contaminant in the input samples is known or required in performing the method.

12. The method of claim 1, wherein the input samples of the set of input samples contains at least one of (i) variable amounts or concentrations of deoxyribonucleic acid (DNA) relative to each other or (ii) different fractions of contaminant DNA relative to each other.

13. The method of claim 1, wherein the collection of intensity signals is from a high-throughput genotyping platform genotyping the input samples using a microarray-based genotyping platform.

14. A computer system comprising:
a memory; and
a processor in communication with the memory, wherein the computer system is configured to perform a method comprising:
obtaining a collection of intensity signals from assays of a set of input samples comprising genetic material;
performing a cross-sample calibration on the intensity signals of the collection of intensity signals based on one or more reference samples, the performing the cross-sample calibration comprising:
constructing a reference signal distribution based on intensity signals of the one or more reference samples; and
for one or more input samples of the set of input samples:
obtaining a respective set of intensity signals, of the collection of intensity signals, corresponding to that input sample, the set of intensity signals corresponding to the input sample comprising (i) a first subset, C, of intensity signals from one or more targeted genomic regions of interest and (ii) a second subset, B, of intensity signals from at least one genomic regions outside the one or more targeted genomic regions of interest; and
calibrating the intensity signals in C based on the reference signal distribution, to produce a respective calibrated set of intensity signals corresponding to the input sample;
determining, for the one or more input samples, and from a respective one or more calibrated sets of intensity signals corresponding to the one or more input samples, a respective at least one aggregated calibrated signal from the one or more targeted genomic regions of interest, wherein the determining produces a collection of aggregated calibrated signals; and
detecting one or more variants in the one or more targeted genomic regions of interest based on the collection of aggregated calibrated signals.

15. The computer system of claim 14, wherein the calibrating of the intensity signals in C, of the set of intensity signals corresponding to the input sample, comprises building a mapping for that input sample based on relations between (i) the intensity signals in B and (ii) the reference signal distribution, wherein the building the mapping comprises defining a mapping function M(x) such that M(x) maps intensity signal x as:
for x existing in B, M(x)=a matching intensity signal from a vector, A, of reference signal intensities, from the reference signal distribution, corresponding to the at least one genomic regions outside the one or more targeted genomic regions of interest;
for x not existing in B but falling between multiple intensity signals in B, M(x)=a linear interpolation based on the M(x) mappings of the multiple intensity signals in B; and
for x not existing in B and not falling within a range of the intensity signals in B, M(x)=an extrapolation based on mappings of highest and lowest quantiles in B.
and wherein the calibrating the intensity signals in C further comprises using the mapping function to map the intensity signals in C to produce the calibrated set of intensity signals corresponding to the input sample.

16. The computer system of claim 14, wherein the obtaining the collection of intensity signals comprises, for the set of input samples, using a set of array hybridization control probes to identify probe hybridization biases by aggregating row-based normalized raw intensity values from the control probes into an aggregated value cₛ, aggregating row-based normalized intensity values from assays targeting human genomic material into an aggregated value xₛ, and determining a contamination factor fₛ as a function of xₛ and cₛ, where fₛ, xₛ and cₛ are determined per input sample.

17. The computer system of claim 16, wherein the determining, for the one or more input samples, and from the respective one or more calibrated sets of intensity signals corresponding to the one or more input samples, the respective at least one aggregated calibrated signal comprises, for an aggregated calibrated signal of the at least one aggregated calibrated signal:
determining a first aggregated signal from a calibrated set of intensity signals corresponding to a targeted region of the input sample; and
using the contamination factor to correct the first aggregated signal and produce a second aggregated signal, wherein the second aggregated signal is output as the aggregated calibrated signal for the targeted region of the input sample.

18. A computer program product comprising:
a computer readable storage medium readable by a processing circuit and storing instructions for execution by the processing circuit for performing a method comprising:
obtaining a collection of intensity signals from assays of a set of input samples comprising genetic material;
performing a cross-sample calibration on the intensity signals of the collection of intensity signals based on one or more reference samples, the performing the cross-sample calibration comprising:
constructing a reference signal distribution based on intensity signals of the one or more reference samples; and
for one or more input samples of the set of input samples:
obtaining a respective set of intensity signals, of the collection of intensity signals, corresponding to that input sample, the set of intensity signals corresponding to the input sample comprising (i) a first subset, C, of intensity signals from one or more targeted genomic regions of interest and (ii) a second subset, B, of intensity signals from at least one genomic regions outside the one or more targeted genomic regions of interest; and
calibrating the intensity signals in C based on the reference signal distribution, to produce a respective calibrated set of intensity signals corresponding to the input sample;
determining, for the one or more input samples, and from a respective one or more calibrated sets of intensity signals corresponding to the one or more input samples, a respective at least one aggregated calibrated signal from the one or more targeted genomic regions of interest, wherein the determining produces a collection of aggregated calibrated signals; and
detecting one or more variants in the one or more targeted genomic regions of interest based on the collection of aggregated calibrated signals.

19. The computer program product of claim 18, wherein the calibrating of the intensity signals in C, of the set of intensity signals corresponding to the input sample, comprises building a mapping for that input sample based on relations between (i) the intensity signals in B and (ii) the reference signal distribution, wherein the building the mapping comprises defining a mapping function M(x) such that M(x) maps intensity signal x as:
for x existing in B, M(x)=a matching intensity signal from a vector, A, of reference signal intensities, from the reference signal distribution, corresponding to the at least one genomic regions outside the one or more targeted genomic regions of interest;
for x not existing in B but falling between multiple intensity signals in B, M(x)=a linear interpolation based on the M(x) mappings of the multiple intensity signals in B; and
for x not existing in B and not falling within a range of the intensity signals in B, M(x)=an extrapolation based on mappings of highest and lowest quantiles in B.
and wherein the calibrating the intensity signals in C further comprises using the mapping function to map the intensity signals in C to produce the calibrated set of intensity signals corresponding to the input sample.

20. The computer program product of claim 18, wherein the obtaining the collection of intensity signals comprises, for the set of input samples, using a set of array hybridization control probes to identify probe hybridization biases by aggregating row-based normalized raw intensity values from the control probes into an aggregated value cₛ, aggregating row-based normalized intensity values from assays targeting human genomic material into an aggregated value xₛ, and determining a contamination factor fₛ as a function of xₛ and cₛ, where fₛ, xₛ and cₛ are determined per input sample, and wherein the determining, for the one or more input samples, and from the respective one or more calibrated sets of intensity signals corresponding to the one or more input samples, the respective at least one aggregated calibrated signal comprises, for an aggregated calibrated signal of the at least one aggregated calibrated signal:
determining a first aggregated signal from a calibrated set of intensity signals corresponding to a targeted region of the input sample; and
using the contamination factor to correct the first aggregated signal and produce a second aggregated signal, wherein the second aggregated signal is output as the aggregated calibrated signal for the targeted region of the input sample.
